(19) United States
(12) Patent Application Publication
(10) Pub. No.: US 2016/0334380 A1
(43) Pub. Date: Nov. 17, 2016

(54) TESTING OF COMPONENTS FOR CONTAMINATIONS

(71) Applicant: Airbus Defence and Space GmbH, Ottobrunn (DE)

(72) Inventors: Georg Wachinger, Ottobrunn (DE); Andreas Helwig, Ottobrunn (DE); Thomas Meer, Ottobrunn (DE); Matthias Geistbeck, Ottobrunn (DE); Alois Friedberger, Ottobrunn (DE); Sebastian Heckner, Ottobrunn (DE)

(21) Appl. No.: 15/151,795

(22) Filed: May 11, 2016

(30) Foreign Application Priority Data
May 11, 2015 (EP) 15167183.1

(51) Int. Cl. G01N 33/00

(52) U.S. Cl. CPC G01N 33/0036

(57) ABSTRACT

A method for testing a component for contamination. The method comprises a heating of a specimen surface of the component under a measuring bell defining a measurement volume, an at least two-time measuring of a contamination of the measurement volume with at least one contaminant, and a purging of the measurement volume with gas. An apparatus for testing a component for contamination is also disclosed. The apparatus comprises a measuring bell configured to form a measurement volume about the component or contacting the component. A heating element is configured to heat at least one specimen surface of the component. A purging unit is configured to purge the measurement volume with gas. A sensor system is configured to measure a contamination of the measurement volume with at least one contaminant.

### CROSS-REFERENCES TO RELATED APPLICATIONS

This application claims the benefit of the European patent application No. 15167183.1 filed on May 11, 2015, the entire disclosures of which are incorporated herein by way of reference.

### BACKGROUND OF THE INVENTION

The present invention relates to a method and to an apparatus for testing components for contamination.

In particular, components that are produced from different substances can be frequently considered as a relatively porous material. This is true, for example, for fiber composite plastics, which comprise fibers and a matrix material, wherein the curing of the material during the manufacture takes place by way of chemical cross-linking. Different substances, which come into contact with such components (such as, for example, humidity, cleaning agents, antifreeze or de-icing agents, operating substances, etc.) can then diffuse into the component, if applicable. The diffusion speed depends, on the one hand, on the properties of the material and, on the other hand, on parameters such as, for example, the ambient temperature, the molecular structure and the polarity of the entering molecule.

Conversely, substances that have entered a component can return as contamination from the interior of the matrix to the surface and, for example, influence subsequent processing steps, such as connections with other materials, bonds or repairs.

During the manufacture and during the employment of components, in particular of elements of fiber composite plastics such as for example carbon fiber-reinforced plastic, an extent of contaminations diffused into the structure is not known in most cases.

The knowledge of such contaminations and their severity, however, can be significant for the further processing such as for example for carrying out structural bonds (e.g., structurally bonded repairs) since contaminations in the material can negatively affect the processed product.

For example, in civil aviation, structural bonds are therefore not permissible for these reasons.

From the publication DE 10 2011 102 055 A1 an apparatus is known by means of which a fiber composite component can be tested for the presence of a plurality of certain contaminants.

### SUMMARY OF THE INVENTION

An object of the present invention is to provide an improved technique with which the components can be tested with respect to contaminations.

A method, according to the invention, serves for testing a component for contamination. It comprises heating a specimen surface of the component under a measuring bell that defines a measurement volume, an at least two-time measuring of a contamination of the measurement volume with at least one contaminant and a purging of the measurement volume with gas.

The measurement of the contamination, according to a method according to the invention, thus takes place several times, at least twice. In the process, at least one current value is measured in each case, which quantifies a contamination of the measurement volume, which, where appropriate, has resulted from a thermal desorption of the contaminant in the measurement volume that was activated through the heating; in the process, the at least one contaminant can be desorbed from the specimen surface and/or through the specimen surface from the interior of the component.

An apparatus according to the invention serves for testing a component for contamination and comprises a measuring bell which is equipped in order to form a (preferentially closed off) measurement volume about the component or contacting the component. An apparatus according to the invention furthermore comprises a heating element which is equipped in order to heat at least one specimen surface of the component, and a purging unit which is equipped in order to purge the measurement volume with gas; in particular, the purging unit can comprise a preferentially controllable gas connection. Finally, an apparatus according to the invention comprises a sensor system which is equipped in order to measure a contamination of the measurement volume with at least one contaminant Depending on a contamination to be detected, the sensor system can comprise, for example, at least one humidity sensor, metal oxide sensor, infrared absorption sensor, ion-mobility spectrometer (IMS), and/or gas chromatography sensor (which can for example comprise an ion-mobility spectrometer as detector). Measuring can comprise determining an absolute or relative quantity of the at least one contaminant in the measurement volume.

“Component” in this document is to mean an element which is suitable for further processing in a manufacturing process or which represents the already finished product. In particular, the term comprises pre-molded elements which for the manufacture of a product can be joined to one another and connected to one another, for example in vehicle construction, in particular, in aircraft construction. A component can be constructed in multiple layers or merely comprise a single layer. A building material unit such as, for example, a board of a fiber composite plastic laminate (e.g., an uncured fiber-resin composite, in particular, a pre-impregnated fiber-matrix mat (a so-called “prepreg”)) is included by the term “component” in this document. Particularly preferred is an embodiment in which the component is a composite component of an aircraft of spacecraft.

The specimen surface can each be a total surface of the component or a local surface portion of the component; testing of the component takes place on the same. Together with the measuring bell and, where appropriate, with a further surface (for example a support), it forms a limit of the (surrounding or contacting) measurement volume. For example, the measuring bell can be put onto the specimen surface of the component or a support in any direction, or it can entirely enclose the component. In particular, the prepositional expression “under a measuring bell” should not be interpreted as indicating a vertical orientation.

The measurement volume can form a geometrical space of any shape (such as, for example, a sphere, a semi-sphere, a cylinder, a truncated cone or a cuboid, in order to mention but a few). In particular, the term “measuring bell” defining the measurement volume must not be understood as a restriction of the geometry of the measurement volume.

According to a preferred embodiment of the present invention, the measuring bell is at least one part of a portable manual apparatus. In particular, it can preferentially have a diameter of at least 10 cm and/or maximally 50 cm, more preferably maximally 20 cm, and/or a mass of maximally 5 kg, more preferably of maximally 1 kg.

Particularly preferred is an embodiment, in which the measuring bell comprises a seal which seals the measurement volume at a transition of the measuring bell to a surface when the measuring bell is placed onto the surface. Such a surface can, for example, comprise a support surface, on which the component lies, and/or it can comprise the specimen surface of the component.

Heating can be effected from any side. In particular, a heating element that is used can be entirely or partly arranged in the measurement volume or outside the same. Particularly preferred is a heating by means of an energy input through an energy-permeable window of the measuring bell. The heating element in this case can comprise, in particular, an infrared radiator.

By purging the measurement volume with gas, gas that is present in the measurement volume is exchanged. In the process, a gas which has the known characteristics is conducted into the measurement volume, in particular, a gas whose contamination with the at least one contaminant does not exceed a known threshold value. According to a preferred embodiment, the gas is air, preferentially synthetic air. Synthetic air offers the advantages of being particularly cost-effective, odorless, non-toxic and non-combustible.

During purging, in particular, contaminant is removed from the measurement volume which where appropriate desorbed from the or through the specimen surface of the component into the measurement volume beforehand. Purging thus prevents a saturation of the measurement volume with the at least one contaminant. In this way, continued desorption of the at least one contaminant into the measurement volume is made possible and precise detecting of a contamination of the component thereby improved.

The present invention thus offers, in particular, the advantage that a thermally activated desorption process can be detected over its course of time. In this way, diffusions in the interior of the component, for example, which result as equalization developments from the desorption, can be taken into account. This makes possible an analysis of the contamination even in inner regions or layers of the component that are distant from the specimen surface. In particular, it can be determined, for example, in this way if a contamination is of such an extent as is critical for certain further processing steps (such as, for example, structural bonding). It can thus be recognized if certain complementary measures (for example drying the component) are required. In this way, a pre-treatment for the further processing steps can be optimized, which reduces costs and work effort.

According to a preferred embodiment, the contaminant comprises humidity. Measuring the contamination in this case can comprise, in particular, a measuring of a relative humidity of a gas (e.g., air) contained in the measurement volume. Alternatively, or additionally, the at least one contaminant can be, for example, ingredients of one or more cleaning and/or antifreeze agents, hydraulic fluid(s), metal oxide(s), lubricants or similar materials.

Preferentially, measuring of a (current) contamination of the measurement volume takes place by means of a sensor system. Such a sensor system can comprise at least one humidity sensor, at least one metal oxide sensor, at least one infrared absorption sensor, at least one gas chromatography ion-mobility spectography sensor (or gas chromatography ion-mobility spectrometer) and/or at least one classic analysis unit. During measuring, an absolute or relative quantity of the at least one contaminant in the measurement volume is preferentially determined.

According to a preferred embodiment version of the present invention, purging is carried out during the heating and/or the (at least two-time) measuring of the contamination. In particular, a desorption process can be activated during a continued purging and detected by way of the multiple measuring of the contamination of the measurement volume; desorbed contaminant is thus preferentially not collected or collected only to a minor degree in the measurement volume in this case. Because of this, a development of the absorption over the time can be directly observed and evaluated for determining a contamination of the component even in surface-distant layers.

Alternatively, or additionally, the purging is carried out during a purging phase, which with respect to time lies between a first and a second activation phase (that is after the first and before the second activation phase). The first and the second activation phase each comprise an at least a one-off measuring of the contamination of the measurement volume with the contaminant (which is then current in each case), wherein the measurement volume during the first and the second activation phase is preferentially closed off. Preferentially, heating the specimen surface takes place at least partly during the first and the second activation phase in which because of this in each case—provided there is a contamination of the component—desorption of the at least one contaminant can be thermally activated.

In this way, a desorbed contaminant can be occasionally collected in the measurement volume and the progressing desorption during the individual activation phases followed. Through the purging between the two activation phases it can be ensured that the starting conditions in both phases coincide; in particular, a diminishing of the absorption as a consequence of a saturation process of the measurement volume with the desorbed contaminant can be prevented. A measuring of the progressing contamination in the closed-off measurement volume makes possible precise detection of the contamination, with which, in particular, in the case of desorption processes that take place slowly, measurement values below a detection threshold can be avoided.

During the purging phase, measuring the contamination can be continued or repeated. The purging phase can thus comprise single or multiple measuring of a contamination of the measurement volume with the contaminant (that is current in each case) during the purging. In this way, the gas in the measurement volume can also be tested during the purging.

In particular, a duration of the purging phase can be controlled dependent on a contamination of the gas. For this purpose, the method can comprise a comparing of a currently measured contamination with a comparison value and a determining that the measured contamination is smaller or equal to a comparison value. The purging can then take place, in particular, by closing a gas connection into the measurement volume, preferentially additionally by closing a gas outlet of the measurement volume.

In this way, it can be achieved without losing time due to an, where appropriate, unnecessarily long continuation of the purging that the gas in the measurement volume at the commencement of the second activation phase has a predetermined quality. Because of this, both suitable desorption preconditions can be created and also in an evaluation of the measured contamination a contamination of the component precisely quantified.

The purging is preferentially controlled automatically. In particular, an employed purging unit preferentially comprises a controller, which automatically controls the start and end of the purging, a conducted gas quantity conducted and/or a purging pressure.

According to a preferred embodiment of a method according to the invention, at least a one-off measuring of the contamination of the measurement volume each before and after an equalization phase of a predetermined duration; the predetermined duration can, for example, be several hours or even days. During the equalization phase, the specimen surface of the component (where appropriate in a portion tested in each case) is temperature controlled to a predetermined temperature which is below an absorption temperature, at which thus no thermal activation of a desorption of the at least one contaminant takes place. The temperatures in this case can be or have been selected, in particular, as a function of the respective contamination and/or the material. For example, the equalization phase can comprise placing the component in a warming chamber or an oven at a temperature which (in particular in the case of humidity contamination) can lie for example in a range of 20°-45° C., more preferably 35° C.-40° C.

Following the equalization phase, renewed heating of the specimen surface of the component preferentially takes place. One or more of the measurements following the equalization phase can be preferentially interrupted by a further purging phase analogously to the ones narrated above. In particular, the measuring following the equalization phase can take place in a third and fourth activation phase which proceed analogously to a first and second activation phase and can be separated from one another by a corresponding purging phase.

As a consequence of a desorption from the specimen surface, a distribution of a contamination in the interior of the component can vary greatly with increasing distance from the surface (for example in various layers of a multi-layered component). The equalization phase in this case can ensure that created concentration differences are offset through diffusion in the interior of the component and an equilibrium with respect to the distribution is thus restored. In particular, it can thereby be achieved that contamination particles located further in the interior of the component diffuse to the surface (in particular to the specimen surface) thus becoming accessible to a desorption through the specimen surface.

According to a preferred embodiment, a method according to the invention comprises a quantifying of a contamination based on the measured contaminations of the measurement volume (where appropriate for example during the activation and/or purging phases); an apparatus according to the invention is analogously equipped preferentially in order to carry out such a quantification. The quantification can concern an inner region of the component (in particular an inner region which towards the outside is closed off by the specimen surface) and/or the specimen surface of the component and/or the entire component.

By means of quantity information thus obtained, a suitability of the component for further processing steps (e.g., bonding) and/or a requirement of taking countermeasures can, for example, be determined. For example, in the case that the at least one contaminant comprises water, it can be determined in this manner if drying is required and where appropriate how long and/or at what temperature such a drying process should take place. Alternatively, or additionally, one or more tolerance limit/s can be determined for the respective contamination. If the determined quantitative contaminations lie below the tolerance limit/s, subsequent drying processes can be omitted.

Quantifying can, in particular, comprise adding of contaminations (i.e., of measurement values which reflect the respective contaminations), such as, for example, adding contaminations determined in a second activation phase (as described above) to a (e.g., maximum) contamination, which was measured in a prior, first activation phase. In this way, the quantity of the contaminant, which up to the respective time was desorbed into the measurement volume, as a whole can be determined, of which during the second activation phase as a consequence of the purging a part is no longer present in the measurement volume.

Particularly preferred is an embodiment in which the quantifying takes place taking into account the respective measurement times at which the respective contaminations were detected.

Alternatively, or additionally, the quantifying can, for example, comprise a determination and analysis of an interpolation function to the measured contaminations as a function of the time; such an analysis can comprise differentiating and/or integrating and/or a determination of extreme values, in particular maxima of the interpolation function.

Quantifying can comprise a comparing of at least one measured contamination, preferentially a course of a measurement series comprising multiple contamination-measurements with one or more value(s) that are stored in a database and/or determined by means of simulation.

Particularly preferred is an embodiment in which the quantifying comprises a generating of a contamination profile which reflects a contamination distribution in a cross section of the component.

A preferred embodiment of an apparatus according to the invention comprises a computer unit which is equipped in order to quantify, based on measured contaminations of the measurement volume, a contamination in the interior of the component and/or on a surface of the component (for example in one of the manners indicated above) and/or determine a contamination distribution in the interior of the component.

According to a preferred embodiment version, the specimen surface of the component is a first specimen surface portion and a method according to the invention comprises a heating of a second specimen surface portion of the component under the (repositioned) measuring bell or under a further measuring bell; in both cases, the respective measuring bell (i.e., the repositioned or the further measuring bell) defines a further measurement volume which adjoins the second specimen surface portion of the component; it is to be understood that the determinations presented above regarding the designations “measuring bell” and “under the measuring bell” also apply with respect to the second specimen surface portion. The method can, furthermore, comprise an at least two-time measurement of a contamination of the further measurement volume with the at least one contaminant and a purging of the further measurement volume with gas.

A measuring bell of a preferred embodiment of the present invention is analogously equipped preferentially in order to be placed one after the other on different portions of the component and/or different components in order to form with the respective contacting portion in each case a (preferentially closed off) measurement volume.

In particular, the component can thus be tested random sample-like on at least one further specimen surface portion according to one or more (where appropriate, different ones) of the methods described here. The results thus obtained, which where appropriate reflect local contaminations in each case can then be quantified for determining a contamination of the entire component (or of a larger portion of a component comprising multiple tested specimen surface portions), and/or a contamination distribution in the component (or the larger portion) can be determined. In this way, (where appropriate utilizing statistical methods), large components can also be tested for contaminations.

According to a preferred embodiment of the present invention, the component contains a fiber composite plastic or the component comprises a fiber composite plastic. In particular, the component can comprise a laminate and/or a prepreg. It can, for example, contain a multi-layered carbon fiber-reinforced plastic (CRP). Such materials can be particularly favorably tested for contaminations with a method according to the invention, respectively by an apparatus according to the invention. Particularly suitable in this case is a cyclical procedure which comprises an equalization phase as narrated and thus takes into account a gradual diffusion of the at least one contaminant from layers of the component located below the surface.

### BRIEF DESCRIPTION OF THE DRAWINGS

In the following, preferred exemplary embodiments of the invention are explained in more detail with the help of drawings. It is to be understood that individual elements and components can also be combined differently than shown.

It shows schematically:

FIG. 1 shows an apparatus according to an exemplary embodiment of the present invention;

FIG. 2 shows an exemplary measurement series as a result of an application of a method according to the invention; and

FIG. 3 shows an exemplary humidity profile of a component according to multiple method cycles.

### DETAILED DESCRIPTION OF THE PREFERRED EMBODIMENTS

FIG. 1 schematically shows (not to scale) an arrangement comprising an embodiment of an apparatus 1 according to the invention.

The apparatus comprises a measuring bell 10, which when contacting a component 20 forms a measurement volume 30. In particular, the measurement volume 30 is enclosed by the measuring bell and a specimen surface 100, which is part of a surface of the component 20.

On its edge, the measuring bell 10 comprises a seal 11 which seals the measurement volume at a transition between the measuring bell 10 and the specimen surface 100.

The component 20 in the shown example comprises a construction by layers (e.g., laminate) with multiple layers 21a, 21b and 21c.

Each of the layers can comprise, for example, a fiber-matrix composite material. Furthermore, the component in its interior comprises a contaminant 22 which has entered various layers of the component interior (i.e., at a component depth).

The apparatus 1 furthermore comprises a heating element 12 which is equipped in order to heat the specimen surface 100. In the shown exemplary embodiment, the heating element is arranged outside the measuring bell 10 and it heats the specimen surface through an energy-permeable window 13 that is located in the measuring bell 10.

A purging unit 14a, 14b comprises a preferentially controllable gas connection 14a and a gas outlet 14b and is equipped in order to pass a predetermined gas (for example synthetic air) through the measurement volume and thereby purge contaminant out of the measurement volume 30 together with the gas.

The apparatus 1 furthermore comprises a sensor system 15 (with one or more sensors), which is equipped in order to measure a contamination of the measurement volume 30 with (at least) the contaminant 22. Finally, the apparatus 1 comprises a computer unit 16 which is connected to the sensor system 15 and equipped in order to quantify, based on contaminations measured by the sensor system 15, a contamination in the interior of the component 20 and/or on the specimen surface and/or determine a contamination distribution in the interior of the component 20.

Using the apparatus, the specimen surface 100 is heated with the help of the heating element 12 which brings about a desorption of the contaminant 22 from a layer 20a near the surface into the measurement volume 30, as indicated by the arrows. The sensor system 15 measures the contamination of the measurement volume resulting from this, preferentially multiple times, and passes the respective measured values on to the computer unit 16.

After a predetermined time (e.g., five minutes), the measurement volume is purged with the help of the purging unit 14a, 14b by passing through gas. Heating is preferentially discontinued in the process. During the purging, measuring of one (in each case current) contamination of the measurement volume 30 with the at least one contaminant 22 can additionally take place. After it has been determined that a predetermined comparison value for a contamination of the measurement volume was undershot, or after expiration of a predetermined time, purging is terminated. Renewed measuring of a contamination of the measurement volume with the contaminant 22 can then take place, after which the component in an equalization phase is temperature-controlled for a predetermined duration to a temperature below desorption temperature before the specimen surface 100 is again heated and a contamination of the measurement volume 30 measured.

Alternatively, the equalization phase can directly follow the purging without a renewed measuring of a contamination of the measurement volume taking place beforehand.

During the equalization phase, parts of the contaminant 22 diffuse out of a deeper (surface-distant) layer 21b of the component 20 diffuse into a layer 20a that is nearer the surface. From there, the contaminant, as a consequence of the thermal activation after the equalization phase, desorbs into the measurement volume where it can be measured. An evaluation of the contamination, taking into account the time of the respective measurement, allows determining a contamination distribution in the interior of the component 20.

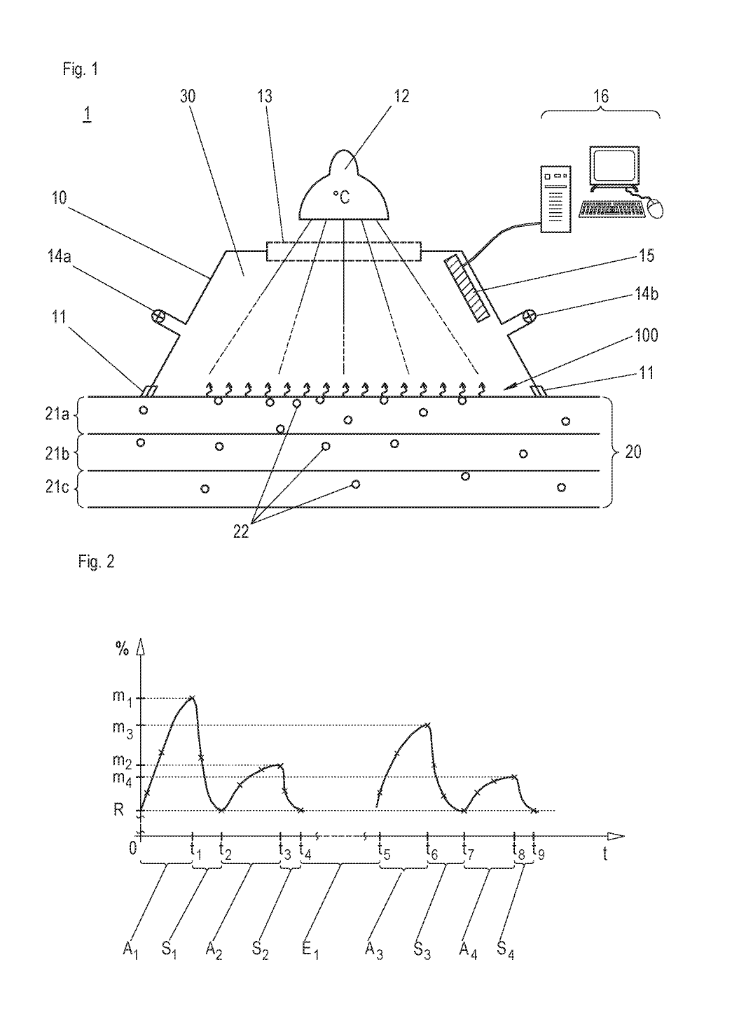

FIG. 2 shows a measurement series as result of carrying out a method according to the invention in an embodiment which is shown as function over the time. Intermediate values were interpolated in the representation. The contaminant can, for example, be water and the measured contamination-measured as relative humidity.

The measurement series reflects a first to fourth activation phase A1, A2, A3, A4, while their respective one desorption of specimen surface was thermally activated. As is evident from the graphic representation of the measurement series, the contamination of the measurement volumes in the different activation phases rose to the respective maximum values m1, m2, m3 and m4.

The respective duration of the individual phases can be suitably selected; for example, for the first and the third activation phases A1 and A3 respectively, 3 to 5 minutes each can be selected and for the second and the fourth activation phase A2 and A4 respectively, 8 to 12 minutes each. According to an exemplary embodiment, the second and the fourth activation phase each take longer than the first and the third activation phases.

The individual activation phases were followed by purging phases S1, S2, S3 and S4, during which the measuring of the contamination was continued. The purging phases were each terminated when the contamination in the measurement volume had fallen to a lower limit value R as a comparison value.

Between the purging phase S2 and the third activation phase A3 an equalization phase E1 took place, during which the component was temperature-controlled to a predetermined temperature which lies below a desorption temperature. According to a special example, the contamination can relate to a humidity, the temperature can have been around 38° C. and the equalization phase E1 can have lasted 19 hours or more; it is to be understood that other contaminations, temperatures and/or durations can also be provided. In the example shown in FIG. 2, contaminant that is present in the interior of the component can diffuse into a layer near the surface during the equalization phase in order to then desorb out of the specimen surface in the third activation phase. This diffusion explains that the contamination-m3 of the measurement volumes measured in the third activation phase is greater in the present case than the contamination-m2 that was measured previously during the second activation phase A2. In particular, the measurement series shows that in the example considered, contaminant before the start of the measurements was also present in deeper (more surface-distal) layers and the corresponding quantity can be determined with the help of the measurement series.

The purging phase S4 can be followed according to a preferred embodiment by at least one further equalization phase which can have the same or another predetermined duration as/than the first equalization phase and following which at least one further activation phase A5 can follow.

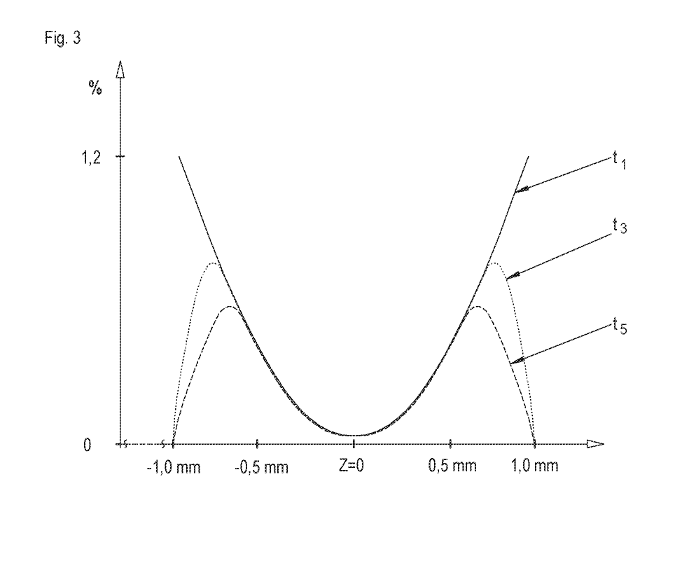

FIG. 3 exemplarily shows three humidity profiles of a 2 mm thick component after, in each case, another activation phase; the center of the component in this case is shown in the zero point here; in the inner layers, the shown component does not have any contamination.

The change of the humidity profile in each case corresponds to the gassed-out volume of the contamination and directly correlates with the measured contaminations. Thus, comparing the measured contaminations with the determined difference profiles, the quantitative contamination and/or the contamination distribution in thickness direction can be directly deduced.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### LIST OF REFERENCE CHARACTERS

1 Apparatus
10 Measuring bell
11 Seal
12 Heating element
13 Window
14a, 14b Purging unit
15 Sensor system
16 Computation unit
20 Component
21a, 21b, 21c Component layer
22 Contaminant
30 Measurement volume
100 Specimen surface
A₁, A₂, A₃, A₄, A₅ Activation phases
S₁, S₂, S₃, S₄ Purging phases
t1 to t9 Time
E₁ Equalization phase
R Comparison value

1. A method for testing a component for contamination, wherein the method comprises:
a heating of a specimen surface of the component under a measuring bell that defines a measurement volume;
an at least two-time measuring of a contamination of the measurement volume with at least one contaminant; and
a purging of the measurement volume with gas.

2. The method according to claim 1, with which the purging takes place during at least one of the heating or the at least two-time measuring steps.

3. The method according to claim 1, wherein the purging takes place during a purging phase, which lies between a first and a second activation phase, and wherein the first and the second activation phase in each case comprise at least a one-off measuring of the contamination of the measurement volume with the contaminant.

4. The method according to claim 3, wherein the purging phase furthermore comprises a one-off or multiple measuring of a contamination of the measurement volume with the contaminant during the purging.

5. The method according to claim 4, with which the purging phase furthermore comprises:
determining that the measured contamination is smaller or equal to a comparison value; and
terminating of the purging by closing a gas connection and a gas outlet.

6. The method according to claim 1, wherein at least one measuring of the contamination takes place before and a measuring of the contamination after an equalization phase, which comprises a temperature-controlling of the specimen surface for a predetermined time to a predetermined temperature below a desorption temperature.

7. The method according to claim 1, which additionally comprises a quantifying of a contamination at least one of in the interior of the component or on the specimen surface of the component, based on the measured contaminations of the measurement volume.

8. The method according to claim 7, with which the quantifying comprises at least one of:
determining a contamination distribution in the interior of the component, or
determining and analysis of an interpolation function to the measured contaminations.

9. The method according to claim 1, wherein the specimen surface is a first specimen surface portion of the component, and wherein the method furthermore comprises:
a heating of a second specimen surface portion of the component under the measuring bell or under a further measuring bell, which in each case defines a further measurement volume;
an at least two-time measuring of a contamination of the further measurement volume with the at least one contaminant; and
a purging of the further measurement volume with gas.

10. The method according to claim 1, wherein the component comprises a fiber composite plastic.

11. The method according to claim 1, wherein the at least one contaminant comprises humidity.

12. The method according to claim 1, wherein the measuring of a contamination of the measurement volume takes place by means of a sensor system, which comprises at least one of a humidity sensor, a metal oxide sensor, an infrared absorption sensor or a gas chromatography ion-mobility spectography sensor.

13. An apparatus for testing a component for contamination, comprising:
a measuring bell configured to form a measurement volume about the component or contacting the component;
a heating element configured to heat at least one specimen surface of the component;
a purging unit configured to purge the measurement volume with gas; and
a sensor system configured to measure a contamination of the measurement volume with at least one contaminant.

14. The apparatus according to claim 13, further comprising a computer unit configured to quantify, based on measured contaminations of the measurement volume, at least one of:
a contamination in the interior of the component,
a contamination on a surface of the component, or
a contamination distribution in the interior of the component.

15. The apparatus according to claim 13, wherein the measuring bell is configured to be placed one after the other on different portions of the component or different components to form a measurement volume in each case with the respective contacting portion.
